(19) United States
(12) Patent Application Publication
(10) Pub. No.: US 2026/0027078 A1
(43) Pub. Date: Jan. 29, 2026

(54) Cyclopropane Fatty Acid Modulators Of Peroxisome Proliferator-Activated Receptors

(71) Applicant: The Regents of the University of California, Oakland, CA (US)

(72) Inventors: Sean Harrison Adams, Sacramento, CA (US); Jean Debédat, Sacramento, CA (US)

(73) Assignee: The Regents of the University of California, Oakland, CA (US)

(21) Appl. No.: 18/998,792

(22) PCT Filed: Jul. 28, 2023
(86) PCT No.: PCT/US2023/071218

§ 371 Date: Jan. 27, 2025

Related U.S. Application Data
(60) Provisional application No. 63/369,711, filed on Jul. 28, 2022.

(51) Int. Cl. A61K 31/20; A61K 9/00; A61K 45/06; A61P 1/16; C07C 53/134

(52) U.S. Cl. CPC A61K 31/20; A61K 9/0056; A61K 45/06; A61P 1/16; C07C 53/134

(57) ABSTRACT

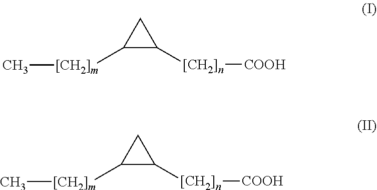

A compound having the structure of Formula I: wherein m=2 and n is an odd-number between 4 and 20, or a derivative thereof, or a salt, or a prodrug thereof, and a compound having the structure of Formula II: wherein m and n are independently an integer between 2 and 20, or a derivative thereof, or a salt, or a prodrug thereof are described. Compositions and methods of use of the compounds for therapeutically modulating peroxisome proliferator-activated receptors (PPARs) are also provided

### BACKGROUND

Initially discovered in the 1950s, cyclopropane fatty acids (CpFAs) are long-chain lipids harboring a cyclopropane ring introduced onto fatty acids' double bonds by bacteria and certain plants. Some CpFAs, such as lactobacillic and dihydrosterculic acids, have been identified in food items (cheese, milk, and fat solely of animals fed silage), and CpFAs have been detected as well as in human adipose, serum and liver, where they are thought to be stored or metabolized via incomplete beta-oxidation. These observations support the idea that gut-derived CpFAs are naturally occurring components of the human lipidome, stored, and released in white adipose tissue similar to “classic” fatty acids.

Little is known about CpFAs' bioactivities, although a recent study demonstrated that lactobacillic acid binds the γ-isoform of peroxisome proliferator-activated receptors (PPARγ), a nuclear receptor involved in lipid anabolism, catabolismor storage, oxidative metabolism, and mitochondrial function.

### SUMMARY

The present disclosure features compounds, compositions, and methods for use of CpFAs having an affinity for binding peroxisome proliferator-activated receptors (PPARs). The PPAR family of nuclear receptors are known to promote metabolic health in liver, adipose tissue, and select other tissues upon activation: e.g., leading to improvement of insulin sensitivity, mitigation of type 2 diabetes and pre-diabetes outcomes, improved lipid homeostasis and reduction in lipotoxicity. For example, PPAR-δ has been shown to promote fatty acid uptake, β-oxidation, and insulin secretion/sensitivity, and PPAR-α has been shown to promote the proper uptake, use, and catabolismof fatty acids. PPAR-γ has been shown to promote proper lipid storage in tissues and support insulin sensitivity and blood sugar control, and is the target of type 2 diabetes drugs, i.e., thiazolidinediones. Concerns about the safety, high drug costs, and costs of monitoring liver function with use of synthetic PPAR ligands has hindered their widespread use and acceptance.

As described in the present disclosure, bacteria-derived CpFAs have been identified. These CpFAs xenolipids display binding affinities toward PPAR α, δ, and/or γ, and may be absorbed and delivered to the liver via the portal vein, to the peripheral tissues via hepatosplanchnic drainage blood or lymphatic lipid transport, and/or released from adipose storage. The CpFAs are also resident metabolites in the intestinal lumen and thus anticipated to have more localized metabolism and activities circa intestinal tissues.

In a one aspect, the present disclosure is directed to a compound of Formula I, or a derivative thereof, or a salt, or a prodrug thereof:

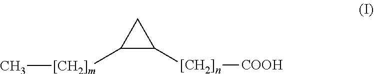

wherein m=2 and n is an odd-number between 4 and 20, for use as a medicament. In some embodiments, the compound can be a medicament for treatment or management of a PPAR-responsive condition. In some embodiments, the medicament can be for treatment or management of weight disorders, insulin resistance, hyperinsulinemia, impaired glucose tolerance, metabolic disorders, diabetes in an obese individual, diabetic complication, a chronic inflammatory disorder, inflammatory bowel disease, ulcerative colitis, Crohn's disease, gastrointestinal disorders, gastroesophageal reflux, diverticulitis, gastrointestinal ulcers, arthritis, rheumatoid arthritis, polyarthritis, asthma, ocular inflammation, thy eye a skin disorder, psoriasis, cancer, liposarcoma, prostate cancer, cervical, breast, multiple myeloma, pancreatic cancer, neuroblastoma, bladder cancer, lipid disorders, hyperlipidemia, dyslipidemia diabetic dyslipidemia, mixed dyslipidemia, hypoalphalipoproteinemia, hypertriglyceridemia, hypercholesterolemia, low high density lipoprotein (HDL), fatty liver disease, non-alcoholic fatty liver disease, non-alcoholic steatohepatitis, cardiovascular disease, and atherosclerosis. In some embodiments, n=9 or n=11. In some embodiments, the compound of Formula I can be cis-11,12-methylene-pentadecanoic acid (cis-11,12-MPD), cis-13,14-methylene-heptadecanoic acid (cis-13,14-MHD), or a combination thereof.

In another aspect, the present disclosure is directed to a method of treating or managing a Peroxisome Proliferator-Activated Receptor (PPAR)-responsive condition in a subject in need thereof, the method including administering to the subject a nutraceutical or pharmaceutical composition containing:
a therapeutically effective amount of an isolated PPAR ligand selected from the group consisting of:
   a compound of Formula I or a derivative thereof, or a salt, or a prodrug thereof:

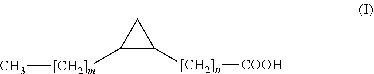

wherein m=2 and n is an odd-number between 4 and 20, and mixtures thereof;
a compound of Formula II or a derivative thereof, or a salt, or a prodrug thereof:

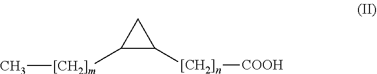

wherein m and n are independently an integer between 2 and 20, and mixtures thereof; and combinations of a compound of Formula I and a compound of Formula II; and a nutraceutically or pharmaceutically acceptable carrier, excipient, adjuvant, or vehicle. In some embodiments, if the PPAR-responsive condition is a PPAR-γ-responsive condition, the compound of Formula II is not lactobacillic acid. In some embodiments, the nutraceutical or pharmaceutical composition includes the compound of Formula I, wherein n=9 or n=11. In some embodiments, the compound of Formula I can be cis-11,12-methylene-pentadecanoic acid (cis-11,12-MPD), cis-13, 14-methylene-heptadecanoic acid (cis-13,14-MHD), or a combination thereof. In some embodiments, the nutraceutical or pharmaceutical composition includes the compound of Formula II, wherein m is 5 or 7 and n is an odd number between 7 and 17. In some embodiments, the compound of Formula II can be cis-9,10-methylene-hexadecanoic acid, lactobacillic acid, dihydrosterculic acid, or a combination thereof. In some embodiments, the nutraceutical or pharmaceutical composition can be administered orally, topically, or parenterally. In some embodiments, the nutraceutical or pharmaceutical composition includes about 1 mg to about 10 g of the isolated PPAR ligand. In some embodiments, the nutraceutical or pharmaceutical composition can be administered once or four times per day. In some embodiments, the method includes administering the isolated PPAR ligand in a daily amount of from about 1 mg to about 10 mg, from about 1 mg to about 30 mg, from about 10 mg to about 100 mg, from about 100 mg to about 500 mg, from about 0.5 g to about 1 g, from about 1 g to about 2 g, from about 2 g to about 4 g, from about 4 g to about 6 g, or from about 6 g to about 10 g. In some embodiments, the PPAR-responsive condition can be a PPARα-, PPARγ-or PPARδ-responsive condition. In some embodiments, the PPAR-responsive condition can be selected from the group consisting of weight disorders, insulin resistance, hyperinsulinemia, impaired glucose tolerance, metabolic disorders, diabetes in an obese individual, diabetic complication, a chronic inflammatory disorder, inflammatory bowel disease, ulcerative colitis, Crohn's disease, gastrointestinal disorders, gastroesophageal reflux, diverticulitis, gastrointestinal ulcers, arthritis, rheumatoid arthritis, polyarthritis, asthma, ocular inflammation, dry eye a skin disorder, psoriasis, cancer, liposarcoma, prostate cancer, cervical, breast, multiple myeloma, pancreatic cancer, neuroblastoma, bladder cancer, lipid disorders, hyperlipidemia, dyslipidemia diabetic dyslipidemia, mixed dyslipidemia, hypoalphalipoproteinemia, hypertriglyceridemia, hypercholesterolemia, low blood high density lipoprotein (HDL), fatty liver disease, non-alcoholic fatty liver disease, non-alcoholic steatohepatitis, cardiovascular disease, and atherosclerosis. In some embodiments, the prodrug form of the PPAR ligand can be administered.

In another aspect, the present disclosure is directed to a pharmaceutical composition for treating or managing a PPAR-responsive condition in a subject in need thereof, the composition containing: an isolated PPAR ligand of Formula I or a derivative thereof, or a salt, or a prodrug thereof:

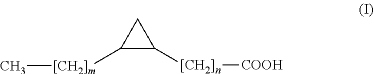

wherein m=2 and n is an odd-number between 4 and 20, and mixtures thereof; and a pharmaceutical carrier, excipient, adjuvant, or vehicle. In some embodiments, n=9 or n=11. In some embodiments, wherein the isolated PPAR ligand of Formula I is cis-11,12-methylene-pentadecanoic acid (cis-11,12-MPD), cis-13,14-methylene-heptadecanoic acid (cis-13,14-MHD), or a combination thereof. In some embodiments, the composition can be formulated for oral, topical, or parenteral administration. In some embodiments, the composition can be formulated as a solid or liquid dosage form. In some embodiments the dosage form can be a tablet, coated tablet, capsule, powder, granulate, solution, dispersion, suspension, syrup, emulsion, or spray. In some embodiments, the pharmaceutical carrier, excipient, adjuvant, or vehicle is selected from the group consisting of diluents, binders, fillers, extenders, humectants, tableting aids, disintegrating agents, absorption accelerators, absorbents, buffering agents, wetting agents, suspending agents, solvents, gel-forming agents, matrix-forming agents, solubilizing agents, emulsifiers, lubricants, encapsulating agents, delayed-release coatings, enteric coatings, sweetening agents, flavoring agents, fragrances, preservatives, and combinations thereof. In some embodiments, the composition further includes a compound of Formula II or a derivative thereof, or a salt, or a prodrug thereof:

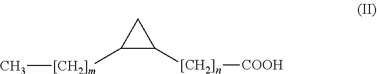

wherein m and n are independently an integer between 2 and 20, and mixtures thereof. In some embodiments, the composition further includes a concomitant component as an additional active pharmaceutical ingredient. In some embodiments, the concomitant component is selected from the group consisting of therapeutic drugs for diabetes, therapeutic drugs for diabetic complications, therapeutic drugs for dyslipidemia, antihypertensive drugs, anti-obesity drugs, therapeutic drugs for fatty liver disease, and combinations thereof.

In another aspect, the present disclosure is directed to a method of treating or managing a PPAR-responsive condition in a subject in need thereof, the method comprising administering to the subject a pharmaceutical composition according to any one of the embodiments of the aspects above.

In another aspect, the present disclosure is directed to a nutraceutical supplement for promoting PPAR modulation in a subject in need thereof, the supplement containing:
an isolated PPAR ligand of Formula I or a derivative thereof, or a salt, or a prodrug thereof:

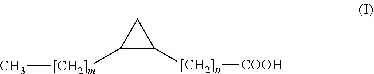

wherein m=2 and n is an odd-number between 4 and 20, and mixtures thereof, and a nutraceutically acceptable carrier, excipient, adjuvant, or vehicle. In some embodiments, n=9 or n=11. In some embodiments, the isolated PPAR ligand of Formula I can be cis-11,12-methylene-pentadecanoic acid (cis-11,12-MPD), cis-13,14-methylene-heptadecanoic acid (cis-13,14-MHD), or a combination thereof. In some embodiments, the supplement can be formulated for oral, topical, or parenteral administration. In some embodiments, the supplement is formulated as a tablet, coated tablet, capsule, aerosol, powder, granulate, suppository, solution, dispersion, suspension, syrup, emulsion, cream, ointment, gel, lotion, or spray. In some embodiments, the supplement further includes a concomitant component as an additional nutraceutical agent. In some embodiments, the concomitant component can be selected from the group consisting of nutraceutical agents for antioxidant support of cells from damage caused by free radicals and oxidative stress; nutraceutical agents to support joint health; nutraceutical agents to improve joint mobility and reduce joint pain; nutraceutical agents to enhance cognitive function, memory, focus, or overall mental performance; nutraceutical agents to promote a healthy gut microbiome; nutraceutical agents to improve digestion and support overall gut health; nutraceutical agents to support healthy cholesterol levels; nutraceutical agents to promote heart health and reduce the risk of heart disease; nutraceutical agents to boost energy levels, enhance vitality, or combat fatigue; nutraceutical agents to support immune system function or strengthen the body's natural defense mechanisms; nutraceutical agents to support weight loss, boost metabolism, or reduce body fat; nutraceutical agents to promote healthy skin, reduce signs of aging, or improve skin elasticity; and combinations thereof. In some embodiments, the supplement can further include a compound of Formula II or a derivative thereof, or a salt, or a prodrug thereof:

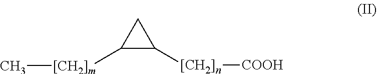

wherein m and n are independently an integer between 2 and 20, and mixtures thereof.

In another aspect, the present disclosure is directed to a method of treating or managing an Angiopoetin-like 4 (Angptl4)-responsive condition in a subject in need thereof, the method comprising administering to the subject a nutraceutical or pharmaceutical composition containing:
a therapeutically effective amount of an isolated cyclopropane fatty acid selected from the group consisting of:
   a compound of Formula I, or a derivative thereof, or a salt, or a prodrug thereof:

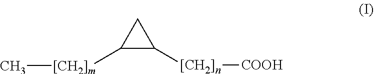

wherein m=2 and n is an odd-number between 4 and 20, and mixtures thereof;
a compound of Formula II, or a derivative thereof, or a salt, or a prodrug thereof:

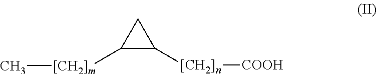

wherein m and n are independently an integer between 2 and 20, and mixtures thereof; and combinations of a compound of Formula I and a compound of Formula II; and a nutraceutically or pharmaceutically acceptable carrier, excipient, adjuvant, or vehicle. In some embodiments, if the PPAR-responsive condition is a PPAR-γ-responsive condition, the compound of Formula II is not lactobacillic acid. In some embodiments, the nutraceutical or pharmaceutical composition includes a compound of Formula I, wherein n=9 or n=11. In some embodiments, the compound of Formula I can be cis-11,12-methylene-pentadecanoic acid (cis-11,12-MPD), cis-13,14-methylene-heptadecanoic acid (cis-13,14-MHD), or a combination thereof. In some embodiments, the nutraceutical or pharmaceutical composition contains the compound of Formula II, wherein m is 5 or 7 and n is an odd number between 6 and 18. In some embodiments, the compound of Formula II can be cis-9, 10-methylene-hexadecanoic acid, lactobacillic acid, dihydrosterculic acid, or a combination thereof. In some embodiments, the nutraceutical or pharmaceutical composition can be administered orally, topically, or parenterally. In some embodiments, the nutraceutical or pharmaceutical composition includes about 1 mg to about 5 g of the isolated cyclopropane fatty acid. In some embodiments, the nutraceutical or pharmaceutical composition is administered once to four times per day. In some embodiments, the method includes administering the isolated cyclopropane fatty acid in a daily amount of from about 1 mg to about 10 mg, from about 1 mg to about 30 mg, from about 10 mg to about 100 mg, from about 100 mg to about 500 mg, from about 0.5 g to about 1 g, from about 1 g to about 2 g, from about 2 g to about 4 g, from about 4 g to about 6 g, or from about 6 g to about 10 g. In some embodiments, the Angptl4-responsive condition can be selected from the group consisting of obesity, insulin resistance, diabetes, hypertriglyceridemia, and hepatic steatosis. In some embodiments, the prodrug form of the cyclopropane fatty acid is administered. In some embodiments, the nutraceutical or pharmaceutical composition further comprises the prodrug form of the compound of Formula I or Formula II.

In another aspect, the present disclosure is directed to a method of treating or managing an Angptl4-responsive condition in a subject in need thereof, the method including administering to the subject a pharmaceutical composition according to any one of the embodiments of the aspects above.

In another aspect, the present disclosure is directed to a functional food or feed composition containing a PPAR-modulating amount of a compound that has the structure of Formula I or a derivative thereof, or a salt, or a prodrug thereof:

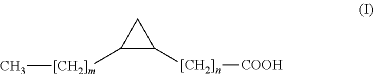

wherein m=2 and n is an odd-number between 4 and 20, and mixtures thereof; and an edible carrier. In some embodiments, n=9 or n=11. In some embodiments, the compound of Formula I can be cis-11,12-methylene-pentadecanoic acid (cis-11,12-MPD), cis-13,14-methylene-heptadecanoic acid (cis-13,14-MHD), or a combination thereof. In some embodiments, the composition includes the prodrug form of the compound of Formula I. In some embodiments, the composition includes about 50 mg to about 2 g of the compound of Formula I per serving. In some embodiments, the composition further includes a compound of Formula II or a derivative thereof, or a salt, or a prodrug thereof:

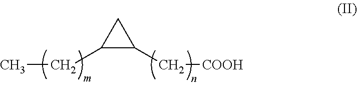

wherein m and n are independently an integer between 2 and 20, and mixtures thereof. In some embodiments, the composition can be a food suitable for human consumption. In some embodiments, the food can be a solid, semi-solid or liquid food stuff. In some embodiments, the composition can be a feed suitable for animal consumption. In some embodiments, the composition further contains an additional nutraceutical agent selected from the group consisting of caffeine, amino acids, creatine, glucosamine, chondroitin, collagen, essential fatty acids, antioxidants, vitamins, minerals, essential oils, extracts, prebiotics, and probiotics, and combinations thereof. In some embodiments, the composition further contains a concomitant component as an active pharmaceutical ingredient. In some embodiments, the concomitant component is selected from the group consisting of therapeutic drugs for diabetes, therapeutic drugs for diabetic complications, therapeutic drugs for dyslipidemia, antihypertensive drugs, anti-obesity drugs, therapeutic drugs for fatty liver disease, and combinations thereof.

In another aspect, the present disclosure is directed to a method of treating or managing an Angptl4-responsive or a PPAR-responsive condition in a subject in need thereof, the method including administering to the subject a nutraceutical supplement or a functional food or feed composition according to any of the embodiments of the aspects above.

It will be apparent that the compounds of Formula I, II, and III, including all subgenera described herein, may have one or more stereogenic centers. The present disclosure contemplates and encompasses each stereoisomer of any compound of encompassed by the disclosure as well as mixtures of said stereoisomers.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. The drawings illustrate generally, by way of example, but not by way of limitation, various embodiments discussed in the present document.

Reference is made to illustrative embodiments that are depicted in the figures, in which:

FIGS. 1A-1C show the chemical structures of example CpFAs ligands of PPARs: (A) cis-11,12-methylene-pentadecanoic acid (cis-11,12-MPD) and (B) cis-13,14-methylene-heptadecanoic acid (cis-13,14-MHD), consistent with the present disclosure.

FIG. 2 shows net uptake/release fluxes of cis-11,12-MPD (left) and cis-13,14-MHD (right) in growing pigs (n=9−12), supporting the idea that CpFAs initially emanate from the gastrointestinal tract in mammals (i.e., from foods and/or microbial metabolism); such a concept aligns with knowledge in the extant literature. PDV, portal drained vein; HQ, hindquarters.

FIG. 3 is an illustration of an in silico binding method using the Schrödinger Molecular Modeling Suite to estimate free energies of ligand-protein complexes from 2D ligand structures and Protein Data Bank crystallized structures of PPAR-α, PPAR-δ, and PPAR-γ.

FIGS. 4 A-B show molecular mechanics/generalized Born surface area (MM-GBSA) binding energies of: (A) PPAR-α, PPAR-δ, and PPAR-γ with high affinity PPARγ antagonists (antago) and agonists (ago) and a representative set of CpFAs consistent with the present disclosure including cis-11,12-MPD and cis-13,14-MHD, from left to right in each panel, GW1929, GW9662, Rosiglitazone, Arachidonic acid, oleic acid, lactobacillic acid, cis-11,12-MPD and cis-13,14-MHD; and (B) binding energies of PPAR-α, PPAR-δ, and PPAR-γ with high affinity PPAR antagonists (*), agonists (**), and a representative set of CpFAs consistent with the present disclosure including cis-11, 12-MPD (C15 odd-chain CpFA) and cis-13,14-MHD (C17 odd-chain CpFA), from left to right, PPAR-α: GW6471, Fenofibrate, Lactobacillic acid, Dihydrosterculic acid, Cyclopropaneoctanoic Acid 2-Hexyl (CPOA2H), C15 odd-chain CpFA, C17 odd-chain CpFA, Oleic acid; PPAR-δ: GSK3787, GW0742, Lactobacillic acid, Dihydrosterculic acid, CPOA2H, C15 odd-chain CpFA, C17 odd-chain CpFA, Oleic acid; and PPAR-γ: GW9662, Rosiglitazone, Lactobacillic acid, Dihydrosterculic acid, CPOA2H, C15 odd-chain CpFA, C17 odd-chain CpFA, Oleic acid). Negative values are reflective of CpFA binding.

FIGS. 5A-C show sigmoidal concentration-response curves for subtype specific synthetic agonists, fatty acids, and a representative number of CpFAs as determined by Time-Resolved Fluorescence Resonance Energy Transfer (TR-FRET) assay: (A) high-affinity PPAR-α agonist GW7416, CpFAs cis-11,12-MPD (C15Δ), cis-13,14-MHD (C17Δ), cis-9, 10-methylene-hexadecanoic acid (C16Δ), lactobacillic acid, and dihydrosterculic acid, and fatty acids palmitoleic acid, oleic acid, erucic acid, and vaccenic acid; (B) high-affinity PPAR-δ agonist GW501516, CpFAs C15Δ, C17Δ, C16Δ, lactobacillic acid, and dihydrosterculic acid, and fatty acids palmitoleic acid, oleic acid, erucic acid, and vaccenic acid; and (C) high-affinity PPAR-δ agonist rosiglitazone, PPAR antagonist T0070907, CpFAs C15Δ, C17Δ, lactobacillic acid, and dihydrosterculic acid, and fatty acids oleic acid, erucic acid, and vaccenic acid.

FIG. 6 shows the results of functional assays measuring Angiopoietin-like Protein 4 (Angptl4) mRNA expression in cultured murine 3T3-L1 cells and the concentration-dependent ability of PPAR antagonists GW6471 (PPAR-α), GSK3787 (PPAR-δ), and T0070907 (PPAR-γ) (0, 0.1, 1, and 10 μM) to reverse the activity of synthetic agonists GW7647 (PPAR-α), GW501516 (PPAR-δ), and rosiglitazone (PPAR-γ), respectively. These results highlight Angptl4 as a PPAR-regulated gene and the value of measuring Angptl4 mRNA as a functional read-out of PPAR activation in this cell model.

FIG. 7A-C show the results of three independent Angptl4 mRNA expression assays (A, B and C) in cultured murine 3T3-L1 cells measuring concentration-dependent induction by synthetic agonists of PPAR-α (GW7647), PPAR-δ (GW501516), and PPAR-γ (ROSI) and a representative number of CpF As at various concentrations (37.5, 75, and 150 μM), consistent with the present disclosure. Oleic, oleic acid; Vaccenic, cis-vaccinic acid; Erucic, erucic acid; Dihydro, dihydrosterculic acid; Lacto, lactobacillic acid; Dihydro, dihydrosterculic acid; C15Δ, cis-11,12-MPD.

### DETAILED DESCRIPTION

The present disclosure features compounds, compositions, and methods for the therapeutic and health-or function-promoting modulation of peroxisome proliferator-activated receptors (PPAR). As used herein, the term “therapeutic” refers to intervention to treat disease or disorders, to provide a beneficial, useful, or favorable result or effect on the body or mind, and the like. A PPAR modulator of the present disclosure can include a cyclopropane fatty acid (CpFA) having the structure of Formula I:

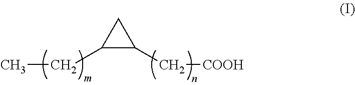

where m=2 and n is an odd-number between 4 and 20, or a derivative thereof, or a pharmaceutically acceptable salt, solvate, complex or prodrug of the compound of Formula I. In some cases, the compound modulates the activity of PPAR α, δ, and/or γ. The compound may be a pan-agonist or modulator. The compound may be an agonist, antagonist or other ligand producing a conformational change in the receptor, inducing recruitment of co-activators or co-repressors of the receptor, or displacing endogenous ligands or co-activators of the receptor. PPAR modulating activity can be assessed using ligand binding studies, such as the studies described in the Examples section. In some examples, n is 9 or 11, or a stereoisomer thereof. For example, the compound of Formula I may include cis-11,12-methylene-pentadecanoic acid (cis-11,12-MPD), cis-13,14-methylene-heptadecanoic acid (cis-13,14-MHD), or a combination thereof.

A PPAR modulator can include CpFAs having the structure of Formula II:

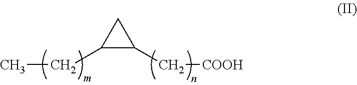

where m and n are independently an integer between 2 and 20, or a derivative thereof, or a derivative thereof, or a pharmaceutically acceptable salt, solvate, complex of prodrug of the compound of Formula II. In some cases, the compound modulates the activity of activity of PPAR α, δ, and/or γ. The compound may be an agonist, antagonist or other ligand producing a conformational change in the receptor, inducing recruitment of co-activators or co-repressors of the receptor, or displacing endogenous ligands or co-activators of the receptor. PPAR modulating activity can be assessed using ligand binding studies, such as the studies described in the Examples section. In some examples, m is 5 or 7 and n is an odd number between 5 and 17, such as 7 or 9. In some examples, the compound of Formula II is cis-9,10-methylene-hexadecanoic acid, lactobacillic acid, dihydrosterculic acid, or a combination thereof. In some examples, such as methods of treating or managing a PPAR-γ-responsive conditions, the PPAR ligand is not lactobacillic acid.

As used herein, “prodrugs” refer to or include compounds that may or may not possess pharmacological activity as such, but which are converted, metabolized, or otherwise bioactivated in the body to form a CpFA that is therapeutically active as a PPAR modulator. As such, in some examples, the prodrug is formulated for and/or administered for the therapeutic modulation of peroxisome proliferator-activated receptors (PPARs). A prodrug of the present disclosure may be a monounsaturated odd-chain fatty acid of Formula III:

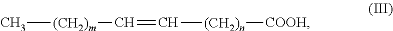

where m=2 and n is an odd-number between 4 and 20, or a derivative thereof or a pharmaceutically acceptable salt, solvate, or complex thereof, that is converted in the body to a cyclopropane fatty acid of Formula I by a bacterial cyclopropane synthase, or where m and n are independently an integer between 2 to 20 or a derivative thereof or a pharmaceutically acceptable salt, solvate, or complex thereof, that is converted in the body to a cyclopropane fatty acid of Formula II by a bacterial cyclopropane synthase. Such fatty acids may occur naturally or be synthetic. In one example of Formula III, n is 7, 9, or 11, or a stereoisomer thereof, or a combination thereof. In some examples, a prodrug of Formula II is octadecenoic acid. In some examples, a prodrug of Formula I is 11-pentadecenoic acid or 13-heptadecenoic acid. In one example of Formula III, n is 9 or 11, or a stereoisomer thereof, or a combination thereof. In some examples, a prodrug according to Formula III is myristoleic acid, palmitoleic acid, cis-vaccenic acid, vaccenic acid, paullinic acid, oleic acid, elaidic acid, 11-eicosenoic acid, erucic acid, brassidic acid, nervonic acid, sapienic acid, or gadoleic acid, petroselinic acid.

The compounds of Formula I and II, and prodrugs thereof, can be used in an isolated form. As used herein, the term “isolated” refers to a compound produced by a process of purification/isolation from a chemical synthesis reaction or purification/isolation from a complex composition, such as a bacterial fermentation or plant extracts. The process of purification/isolation provides an effective amount of the compound, or prodrug thereof, for therapeutic use. The purified/isolated preparation can consist essentially of or consist of the compound of Formula I and/or II, thereby creating a new composition that exhibits markedly different characteristics than the fermentation product or plant extracts, for example. In some examples, purification/isolation improves the formulation properties of the compound (e.g., allows for improved tableting) or the bioavailability of the compound, as compared with the unpurified starting composition.

The compounds of Formula I and II, and prodrugs thereof, can be used in various combinations. For example, combinations for use in the therapeutic modulation of PPARs include two or more species encompassed by Formula I, such as cis-11, 12-MPD and cis-13,14-MHD, two or more species encompassed by Formula II, such as at least two of cis-9,10-methylene-hexadecanoic acid, lactobacillic acid, and dihydrosterculic acid, at least one species encompassed by Formula I and at least one species encompassed by Formula II, two or more species encompassed by Formula III, such as vaccenic acid and oleic acid, at least one species encompassed by Formula I and/or Formula II and/or Formula III. The combination can be formulated by mixing isolated compounds and exhibit markedly different characteristics than the isolated compounds. In some examples, the combination exhibits an improved therapeutic outcome or a synergistic effect.

Suitable pharmaceutically acceptable salts include metal salts, such as for example, aluminum, alkali metal salts such as lithium, sodium or potassium, alkaline metal salts such as calcium or magnesium and ammonium or substituted ammonium salts.

A compound as described herein may be a medicament. As used herein, a “medicament” refers to or includes a compound according to Formula I and/or Formula II, in any form suitable to be used for a medical purpose, e.g., in the form of a medicinal product, a pharmaceutical preparation or product, a nutraceutical or supplement formulation, a veterinary formulation, a dietary product, a functional food or feed composition or supplement, or the like, and/or to be used for the prevention, treatment or management of a specific medical condition for which modulation of one or more PPARs is beneficial. The compound may have an affinity for at least one of the PPAR isoforms α, y or δ, such as a human PPAR isoform, that confers efficacy for the prevention, treatment, or management of weight disorders, insulin resistance, hyperinsulinemia, impaired glucose tolerance, metabolic disorders, diabetes in an obese individual, diabetic complication, a chronic inflammatory disorder, inflammatory bowel disease, ulcerative colitis, Crohn's disease, gastrointestinal disorders, gastroesophageal reflux, diverticulitis, gastrointestinal ulcers, arthritis, rheumatoid arthritis, polyarthritis, asthma, ocular inflammation, dry eye a skin disorder, psoriasis, cancer, liposarcoma, prostate cancer, cervical, breast, multiple myeloma, pancreatic cancer, neuroblastoma, bladder cancer, lipid disorders, hyperlipidemia, dyslipidemia diabetic dyslipidemia, mixed dyslipidemia, hypoalphalipoproteinemia, hypertriglyceridemia, hypercholesterolemia, low blood high density lipoprotein (HDL), fatty liver disease, non-alcoholic fatty liver disease, non-alcoholic steatohepatitis, cardiovascular disease, and atherosclerosis. In some examples, the affinity for one or more PPAR isoforms α, y or δ, confers efficacy for the prevention, management, or treatment of metabolic syndrome, a dyslipidemic condition, e.g., hypertriglyceridemia (HTG), elevated triglyceride levels, LDL cholesterol levels, and/or VLDL cholesterol levels, obesity or an overweight condition, a fatty liver disease, peripheral insulin resistance and/or a diabetic condition, type 2 diabetes, or the reduction of plasma insulin, blood glucose and/or serum triglycerides, and combinations of these conditions. Thus, in specific examples, the medicament is for the prevention, treatment, or management of diabetes, fatty liver disease, non-alcoholic fatty liver disease, or non-alcoholic steatohepatitis. The present disclosure encompasses the use of a compound according to Formula I and II for other uses, including the manufacture of the medicament.

The present disclosure describes pharmaceutical compositions comprising a compound of Formula I and/or Formula II as an active ingredient, and/or a compound of Formula III as a prodrug thereof. As used herein, a “pharmaceutical composition” (or “preparation” or “product”) includes a compound of Formula I or Formula II as described above, as an active ingredient in any form that is suitable for a medical purpose. The composition may include a pharmaceutically acceptable carrier, excipient, adjuvant, or vehicle. As used herein, “carrier” refers to diluents, disintegrants, precipitation inhibitors, surfactants, glidants, binders, lubricants, and other excipients and vehicles in which or with which the compound is administered. The route of administration typically determines the type of carrier. A pharmaceutical composition may be formulated for oral administration to a human or an animal, however, examples are not so limited. The pharmaceutical composition may be formulated for administration through any other route where the active ingredients may be efficiently absorbed and utilized, e.g., intravenously, parenterally, subcutaneously, intramuscularly, orally, intranasally, ocularly, rectally, vaginally, or topically. The pharmaceutical composition can be formulated for local or systemic effect, or a combination thereof. The compounds and compositions may be formulated as tablets, coated tablets, capsules, aerosols, powders, granulates, suppositories, solutions, dispersions, suspensions, syrups, emulsions, creams, ointment, gels, lotions, and sprays, using formulation aids. Non-limiting examples of formulation aids include diluents, binders, fillers, extenders, humectants, tableting aids, disintegrating agents, absorption accelerators, absorbents, buffering agents, wetting agents, suspending agents, solvents, gel-forming agents, matrix-forming agents, solubilizing agents, emulsifiers, lubricants, encapsulating agents, delayed-release coatings, enteric coatings, sweetening agents, flavoring agents, fragrances, preservatives, pH modifiers, antioxidants, stabilizing agents, hard fats, or mixtures thereof. The composition may include the compound within a range of about 0.1% to about 99.9% by weight, such as about 10% to 50% by weight or about 30% to about 95% by weight.

The amount of Formula I, II, and/or III present in the pharmaceutical composition or unit dose thereof, can be the amount capable of providing the medicinal benefit in a single dose or over multiple doses. For example, the amount of Formula I, II, and/or III in a unit dose can be from about 1 mg to about 10 mg, from about 1 mg to about 30 mg, from about 10 mg to about 100 mg, from about 100 mg to about 500 mg, from about 0.5 g to about 1 g, from about 1 g to about 2 g, from about 2 g to about 4 g, from about 4 g to about 6 g, or from about 6 g to about 10 g.

A pharmaceutical composition of the present disclosure can include one or more further active ingredients in addition to the compound of Formula I, II and/or III, as a concomitant component. The dose of the concomitant component, or ratio of two or more concomitant components can be determined based on clinically suitable protocols. The addition of the concomitant component can enhance the action of compound of Formula I, II and/or III of the present disclosure or the concomitant component and/or permit a reduction in the dose of the compound of Formula I, II and/or III, or the concomitant component thereby avoiding adverse or undesirable side effects as compared to a single drug administration while achieving the desired therapeutic outcome. In some examples, the concomitant component is a therapeutic drug for diabetes, therapeutic drug for diabetic complications, therapeutic drug for dyslipidemia, antihypertensive drug, anti-obesity drug, therapeutic drug for fatty liver disease, or a combination thereof. The concomitant component can be a low-molecular-weight compound, a high-molecular-weight protein, polypeptide, antibody, or vaccine.

The therapeutic drug for diabetes can include one or more insulin preparations, α-glucosidase inhibitors, biguanides (e.g., phenformin, metformin, buformin or a salt thereof (e.g., hydrochloride, fumarate, succinate), secretagogues (e.g., tolbutamide, glibenclamide, gliclazide, chlorpropamide, tolazamide, acetohexamide, glyclopyramide, glimepiride, glipizide, glybuzole, repaglinide, nateglinide, mitiglinide or calcium salt hydrate thereof), GLP-1 receptor agonists, dipeptidyl-peptidase IV inhibitors, β3 agonists, amylin agonists, phosphotyrosine phosphatase inhibitors, gluconeogenesis inhibitor (e.g., glycogen phosphorylase inhibitor, glucose-6-phosphatase inhibitor, glucagon antagonist), sodium-glucose cotransporter inhibitors, 11β-hydroxysteroid dehydrogenase inhibitors, adiponectin or agonist thereof, IKK inhibitors, leptin resistance-improving drug, somatostatin receptor agonists, and glucokinase activators.

The therapeutic drug for diabetic complications can include, for example, one or more of aldose reductase inhibitors (e.g., tolrestat, epalrestat, zenarestat, zopolrestat, minalrestat, fidarestat, CT-112, ranirestat), neurotrophic factors (e.g., NGF, NT-3, BDNF), neurotrophic factor production-secretion promoters, PKC inhibitors (e.g., ruboxistaurin mesylate), AGE inhibitors (e.g., pimagedine, pyratoxanthine, N-phenacylthiazolium bromide, pyridorin, pyridoxamine), active oxygen scavengers (e.g., thioctic acid), cerebral vasodilators (e.g., tiapuride, mexiletine), somatostatin receptor agonists and apoptosis signal regulating kinase-1 (ASK-1).

The therapeutic drug for dyslipidemia can include, for example, one or more of HMG-CoA reductase inhibitors (e.g., pravastatin, simvastatin, lovastatin, atorvastatin, fluvastatin, lipantil, itavastatin, rosuvastatin or a salt thereof, fibrate compounds (e.g., bezafibrate, beclobrate, binifibrate, ciprofibrate, clinofibrate, clofibrate, clofibric acid, etofibrate, fenofibrate, gemfibrozil, nicofibrate, pirifibrate, ronifibrate, simfibrate, theofibrate), ezetimibe, squalene synthase inhibitors, ACAT inhibitors (e.g., avasimibe, eflucimibe), anion exchange resins (e.g., colestyramine), probucol, nicotinic acid drugs (e.g., nicomol, niceritrol), omega-3-fatty acids, niacin, ethyl icosapentate, phytosterol (e.g., soysterol, γ-oryzanol) and Proprotein convertase subtilisin/kexin type 9 (PCSK9) inhibitors.

The antihypertensive drug can include, for example, one or more of angiotensin converting enzyme inhibitors (e.g., captopril, enalapril, delapril), angiotensin II antagonists (e.g., losartan, eprosartan, valsartan, telmisartan, irbesartan, tasosartan, olmesartan medoxomil), calcium antagonists (e.g., manidipine, nifedipine, nicardipine, amlodipine, efonidipine), potassium channel openers (e.g., levcromakalim), and clonidine.

The antiobesity drug can include, for example, one or more of antiobesity drugs acting on the central nervous system (e.g., Naltrexone-bupropion, dexfenfluramine, fenfluramine, phentermine, sibutramine, anfepramone, dexamphetamine, mazindol, phenylpropanolamine, clobenzorex; MCH receptor antagonists, neuropeptide Y antagonists, cannabinoid receptor antagonists, ghrelin antagonist; 11β-hydroxysteroid dehydrogenase inhibitors, pancreatic lipase inhibitors (e.g., orlistat, cetilistat), β3 agonists, anorectic peptides (e.g., leptin, CNTF (ciliary neurotrophic factor)), cholecystokinin agonists (e.g., lintitript), liraglutide and feeding deterrents.

The present disclosure describes nutraceutical compositions, such as supplements, functional food or feed compositions, food or feed supplements, food or feed additives, enteral nutrition compositions, veterinary formulations, or other nutraceutical preparations, comprising a compound of Formula I and/or Formula II as an active ingredient, and/or a compound of Formula III as a prodrug thereof. As used herein, “nutraceutical” refers to a product derived from a food source and/or added to a food source, which provides a medicinal, health or functional benefit. The nutraceutical aids in the prevention, management, or treatment of any one of the conditions described above, or a combination of conditions. Nutraceutically acceptable carriers, mediums, diluents, and excipients include those suitable for human or animal consumption and/or used in the food industry. The present disclosure describes a functional food or feed composition, such as a food or feed supplement, a food or feed additive, or a nutraceutical preparation, comprising a compound of Formula I and/or Formula II, or prodrug thereof. “Food (or feed) composition” refers to a liquid, semi-solid or solid food product or nutritional composition, suitable for human or animal consumption, including free-flowing and semi-solid beverage compositions. For example, the feed composition may be provided to livestock or companion animals. Companion animals may include, but are not limited to, cats, dogs, horses, and other mammals. A functional food or feed composition of the present disclosure may be provided and/or sold as a food, feed, or beverage product labeled by intended therapeutic use or function, such as “for promoting liver health”, “for regulating metabolism”, or others as described herein.

The amount of Formula I, II, and/or III present in the nutraceutical composition, e.g., the functional food or feed composition, is an amount capable of providing the medicinal benefit in a single serving or over multiple servings. For example, the amount of Formula I, II, and/or III in a single serving can be from about 1 mg to about 10 mg, from about 10 mg to about 100 mg, from about 100 mg to about 500 mg, from about 0.5 g to about I g, from about 1 g to about 2 g, from about 2 g to about 4 g, from about 4 g to about 6 g, or from about 6 g to about 10 g.

In some examples, a nutraceutical composition of the present disclosure includes one or more concomitant components as additional nutraceutical agents. Examples include caffeine, amino acids, creatine, glucosamine, chondroitin, collagen, essential fatty acids, antioxidants, vitamins, Coenzyme Q10, minerals, essential oils, extracts (e.g., herbal or fruit extracts), prebiotics, and probiotics. The additional nutraceutical agent can be included for antioxidant support of cells from damage caused by free radicals and oxidative stress, to support joint health, improve joint mobility and reduce joint pain, to enhance cognitive function, memory, focus, and overall mental performance, to promoting a healthy gut microbiome, improve digestion, and support overall gut health, to support healthy cholesterol levels, promote heart health, and reduce the risk of heart disease, to boost energy levels, enhance vitality, and combat fatigue, to support immune system function and strengthen the body's natural defense mechanisms, to support weight loss, boost metabolism, and reduce body fat, to promote healthy skin, reduce signs of aging, and improve skin elasticity.

Exemplary essential fatty acids include, but are not limited to, polyunsaturated fatty acid (PUFA), e.g., an omega-3 fatty acid selected from alpha-linolenic acid (18:3, ALA), stearidonic acid (18:4), eicosatetraenoic acid (20:4), eicosapentaenoic acid (20:5; EPA), docosatetraenoic acid (22:4), n-3 docosapentaenoic acid (22:5; n-3DPA), and docosahexaenoic acid (22:6; DHA), e.g., an omega-6 fatty acid selected from linoleic acid (18:2), gamma-linolenic acid (18:3), eicosadienoic acid (20:2), dihomo-gamma-linolenic acid (20:3), arachidonic acid (20:4), docosadienoic acid (22:2), adrenic acid (22:4), and n-6 docosapentaenoic acid (22:5), e.g., an omega-9 fatty acid such as mead acid (20:3). Essential fatty acids can be provided in the form of seafood-derived oil, plant-derived oil, and algae-derived oil. The derived oil may also be hydrogenated or partially hydrogenated.

In some examples, a nutraceutical composition of the present disclosure includes at least one pharmaceutically acceptable agent, such as the concomitant components described above for pharmaceutical compositions.

The present disclosure describes methods including administering to a subject a therapeutically effective amount of a compound of Formula I and/or Formula II, as an active ingredient, or sufficient amount of a prodrug thereof to provide a therapeutical effect after conversion to a compound of Formula I and/or Formula II. As used herein, a “therapeutically effective amount” refers to or includes an amount of a compound according to Formula I and/or Formula II that will lead to the desired therapeutic, health or functional effects, i.e., an amount that is effective to achieve its intended purpose. While individual subject needs may vary, the dosage regimen for treating a condition with the disclosed product of this disclosure is selected in accordance with a variety of factors, including the type, age, weight, sex, diet, and medical condition of the subject. The subject may be an adult, a juvenile, an infant, or a fetus. The therapeutically effective amount of the compound may be included in a pharmaceutical composition as well as a food stuff, a food supplement, a nutritional supplement, or a dietary product. In some examples, a therapeutically effective amount is a “prophylactically effective amount” whereby a pathological condition, or sign or symptom thereof, is prevented in a subject at risk of developing the condition. “Treatment” or “management” includes any therapeutic application that may benefit a human or non-human mammal. “Health or function” refers to desired physiological outcomes even in the absence of overt disease or pre-disease. Both human and veterinary treatments are within the scope of the present disclosure. Treatment may be in respect of an existing condition or it may be prophylactic, for example, preventative. The term “chronic treatment” refers to or includes treatment that continues for weeks or years.

The route of administration depends on various factors such as characteristics of the compound, such as onset of action and bioavailability, the formulation of the compound, the intended therapeutic effect, and the condition of the subject. In some examples, a method of the present disclosure includes oral, ocular, otic (aural), sublingual, buccal, inhalation, intranasal, parenteral, intravenous (IV), intramuscular (IM), subcutaneous (SC), intradermal (ID), topical (e.g., for administration to internal tissue surfaces (e.g., gastrointestinal surfaces) and/or external tissue surfaces (e.g., skin, eyes, ears), transdermal, rectal, vaginal, epidural, and intrathecal route of administration, or a combination of routes.

The present disclosure describes methods including administering to a subject a therapeutically effective amount of a compound of Formula I and/or Formula II, as an active ingredient, or sufficient amount of a prodrug thereof to provide a therapeutic effect after conversion to a compound of Formula I and/or Formula II, and a second active agent, i.e., the concomitant component, as described above. Formula I, II, and/or III and the concomitant component can be administered simultaneously or in a staggered manner to the subject. The compound of Formula I, II, and/or III and the concomitant component can be administered to the subject as two or more formulations or preparations each containing the compound(s) or a single preparation containing both active ingredients.

Methods of the present disclosure may include regulating metabolism and/or promoting health. As one example, a method of treating (or preventing or managing) diabetes mellitus, metabolic syndrome, secondary diabetes, pancreatic, extrapancreatic/endocrine or drug-induced diabetes, or exceptional forms of diabetes (e.g., lipoatrophic, myatonic or a disease caused by disturbance of the insulin receptors) is described. The method includes administering a compound of Formula I and/or Formula II, or prodrug thereof, to a subject in need thereof. A subject in need of treatment may be a subject identified as having metabolic syndrome and characterized by hyperinsulinemia, insulin resistance, obesity, glucose intolerance, hypertension, abnormal blood lipids, or dyslipidemia, for example. The method may include treatment and/or prevention of peripheral insulin resistance and/or treatment and/or prevention of type 2 diabetes, or the reduction of plasma insulin, blood glucose, glycated hemoglobin and/or serum triglycerides.

Another example method includes a method of treating (or preventing) obesity or managing a weight control condition comprising administering a compound of Formula I and/or Formula II, or prodrug thereof, to a subject in need thereof. A subject in need of treatment may be obese, in need of weight management, and/or may have an increased insulin resistance, for example.

Another example method includes a method of treating (or managing or preventing) elevated blood lipids in humans comprising administering a compound of Formula I and/or Formula II, or prodrug thereof, to a human in need thereof. The human may have a dyslipidemic condition, e.g., hypertriglyceridemia (HTG), elevated triglyceride levels, LDL cholesterol levels, and/or VLDL cholesterol levels.

Another example method includes a method of treating (or managing or preventing) a liver disease, such as a non-alcoholic liver disease, e.g., non-alcoholic fatty hepatitis (NASH), or non-alcoholic fatty liver disease (NAFLD), comprising administering a compound of Formula I and/or Formula II, or a prodrug thereof, to a subject in need thereof. A subject in need of treatment and/or prevention may be overweight or obese, and/or have diabetes, hyperlipidemia, metabolic syndrome, or a poor diet.

A suitable daily dosage of a compound of Formula I, Formula II, or prodrug thereof, according to the present disclosure, for treatment, management, and/or prevention of the conditions listed above, may be within the range of about 1 mg to about 10 g, about 1 mg to about 30 mg, about 30 mg to about 100 gm, about 100 mg to about 1 g, or about 1 g to about 10 g of the compound daily. The daily dosage may be about 1 mg to about 3 g, about 1 mg to about 10 g, about 1 to about 30 mg, about 50 mg to about 1 g, about 10 mg to about 2 g, about 50 mg to about 500 mg, about 50 mg to about 200 mg, about 100 mg to about 1 g, about 100 mg to about 500 mg, or about 100 mg to about 250 mg. The dosage administered will vary with the compound employed, mode of administration, treatment desired and disorder indicated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The compound, medicament, nutraceutical formulation, veterinary formulation, food, or feed composition and/or the pharmaceutical composition of the present disclosure may be administered in accordance with a regimen of from 1 to 10 times per day, such as once, twice, three times, or four times per day. For oral and parenteral administration to human patients, the daily dosage level of the agent may be in single or divided doses.

### EXAMPLES

The following examples are provided for illustrative purposes only and not to limit the scope of the claims provided herein.

### Example 1

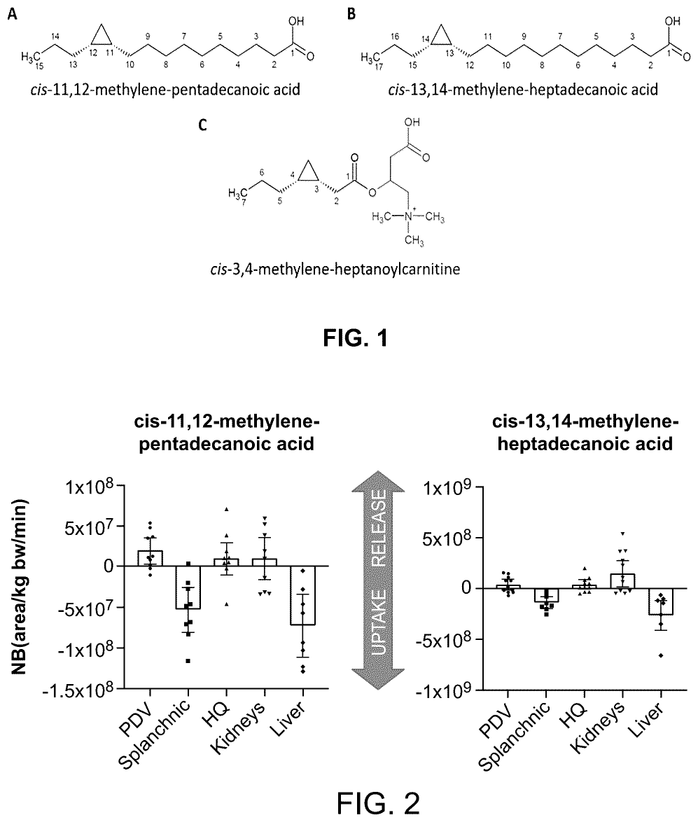

This example describes newly identified ligands for PPAR nuclear receptors: cis-11,12-methylene-pentadecanoic acid (cis-11,12-MPD) and cis-13,14-methylene-heptadecanoic acid (cis-13,14-MHD). Two odd-chain, long-chain fatty acids containing an unusual cyclopropane ring (FIGS. 1A and 1B), which have not been previously reported were deduced and predicted from the structure of cis-3,4-methylene-heptanoylcarnitine (cis-3,4-MHC, FIG. 1C); the latter molecule has been reported in the scientific literature previously. Without being bound by theory, cis-3,4-MHC is believed to be the derivative of cis-11,12-MPD and cis-13,14-MHD, produced through several cycles of beta-oxidation. Previous published studies showed that serum levels of this medium-chain acylcarnitine significantly increased during an exercise bout and reduced at rest by a weight loss and exercise intervention in insulin-resistant sedentary women.

Long-chain cyclopropane fatty acids (CpFAs) such as cis-11,12-MPD and cis-13,14-MHD may be present in mammalian gut and in the case of cis-11,12-MPD and cis-13,14-MHD believed to be primarily derived from microbial metabolism of odd-chain fatty acid precursors. This conclusion stems from observations described for different even-chain CpFAs described elsewhere in the scientific literature. Odd-chain CpF As of the present disclosure have utility body-wide to improve metabolic health, but notably in liver, as the liver is an early tissue that interacts with gut-derived signals. Based on initial results, cis-11,12-MPD and cis-13,14-MHD may be metabolized in the liver, but also reach other organ sites including adipose tissue in which they can be stored. As with other CpFA PPAR modulators, actions of cis-11,12-MPD and cis-13,14-MHD to prevent or treat disease, or improve health or function, could also take place in the gastrointestinal tract.

### Example 2

In pilot studies using archived samples, the presence of cis-11,12-MPD and cis-13,14-MHD in piglet feces was confirmed by initially synthesizing authentic chemical standards using standard organic chemistry methods, and then using the standards in a mass spectrometry library. In follow-on collaborative pilot studies in a tissue-specific blood catheterization model, the portal vein blood kinetics of cis-11,12-MPD and cis-13,14-MHD in the blood of growing pigs were consistent with the gut as a site of origin to enter the body (FIG. 2), consistent with the knowledge in the extant literature supporting that in mammals, CpFAs emanate from gut sources.

### Example 3

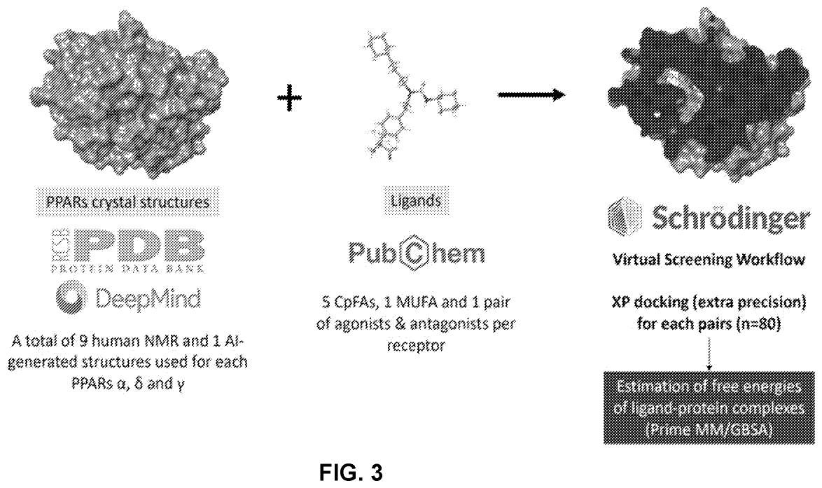

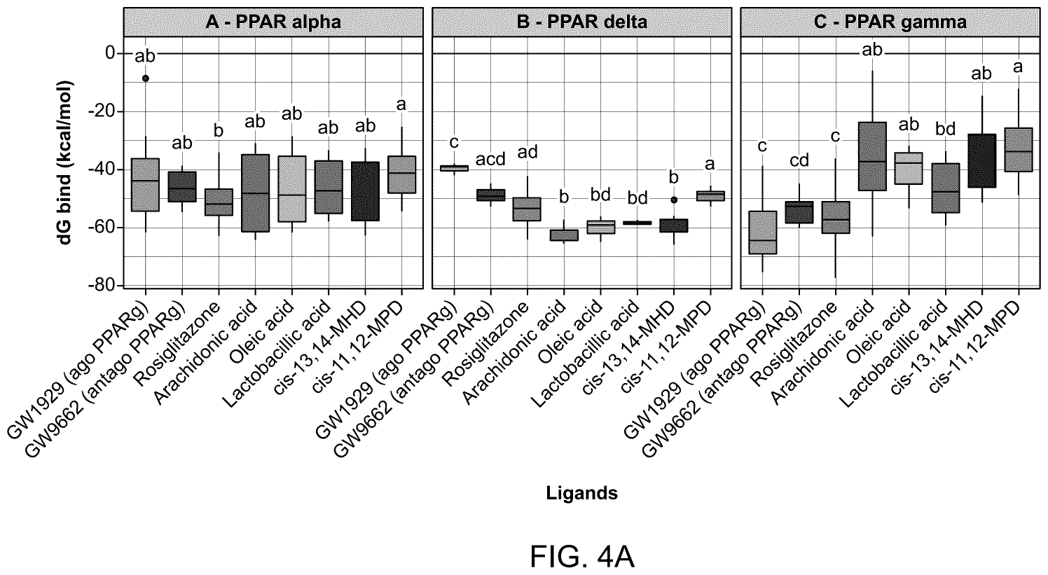

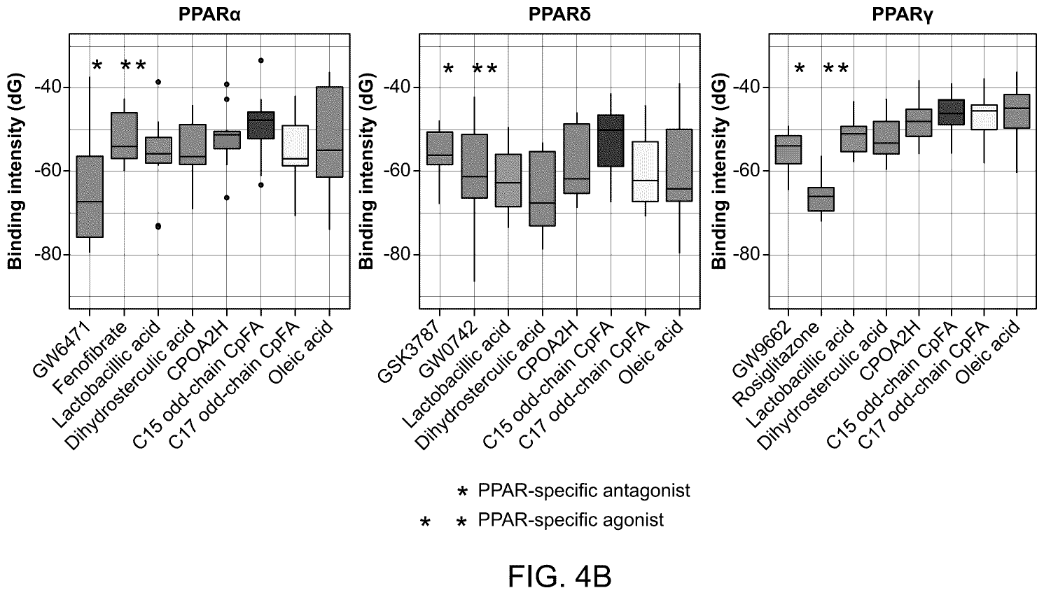

The Schrödinger molecular modeling suite (version 12.8.117 release 2021-2) was used to evaluate binding of cis-11, 12-MPD and cis-13,14-MHD with PPAR family member proteins (FIG. 3). Molecular modeling is a commonly used in silico tool used to evaluate the binding potential of molecules to targets relevant to medicine and health, including nuclear receptors, cell surface receptors, channels, and enzymes. Positive controls included well-established PPAR binding molecules: rosiglitazone and GW1929 (PPAR-γ activators), the drug GW9662 (PPAR-γ antagonist), and several fatty acids known or proposed in the literature to bind to PPARs (arachidonic, oleic, and lactobacillic acids). The results obtained as described below revealed that CpFAs, including cis-11,12-MPD and cis-13,14-MHD avidly bind PPAR-α, PPAR-δ, and PPAR-γ (FIG. 4A and B). While cis-11,12-MPD and cis-13,14-MHD bound all forms of the PPARs, the binding energy patterns indicated the strongest binding to PPAR-γ (−60 kcal/mol) when compared to PPAR-α and PPAR-γ (−40 kcal/mol) (a lower negative binding energy indicates a higher binding affinity). In some cases, the binding energies were similar to those of PPAR-selective pharmacologic ligands, namely fenofibrate, GW501516, and rosiglitazone for PPAR α, δ and γ, respectively. The highest binding affinity was determined to be toward PPAR δ, followed by PPAR α, and finally the γ subunit. These results also show that CpFAs lactobacillic acid and dihydrosterculic acid had unexpected binding affinities for PPAR-α and PPAR-δ, a novel observation. Previous reports implicated only the PPAR-γ isoform for lactobacillic acid.

This study revealed that cis-11,12-MPD and cis-13,14-MHD, since they are natural ligands, may have an improved safety profile over PPAR-targeting drugs. In addition, cis-11,12-MPD and cis-13,14-MHD are likely to impinge on hepatic metabolism and liver health more specifically than other tissues, when compared to current pharmaceuticals targeting the PPAR family. Thus, the odd-chain CpFAs are strong candidates for therapeutic targeting of diseases associated with perturbed liver metabolism. Since cis-11,12-MPD and cis-13,14-MHD can bind PPAR-α, PPAR-δ, and PPAR-γ, they might modulate several metabolic pathways at once; which may be advantageous over the highly isoform specific pharmaceuticals currently available. Finally, PPAR-modulating CpFAs, including cis-11,12-MPD and cis-13,14-MHD, should have advantage over typical oral PPAR-targeting drugs, since the latter are not formulated to treat or prevent conditions of the intestinal tract whereas CpFAs are typically found or introduced at this site.

### Example 4

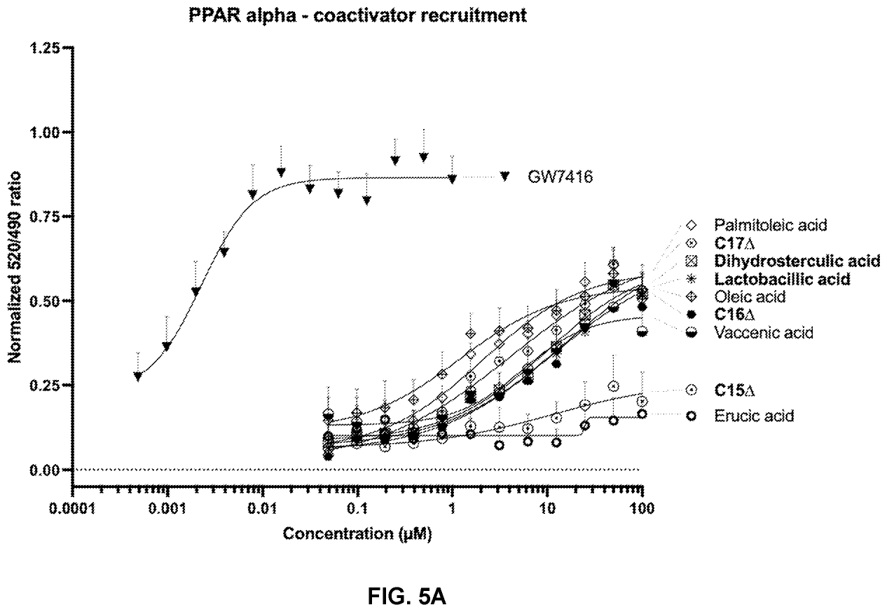

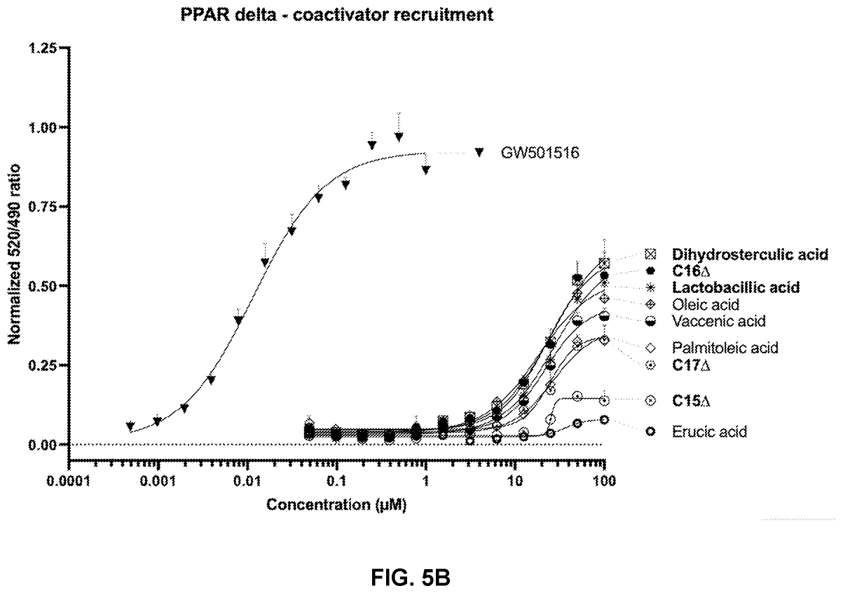

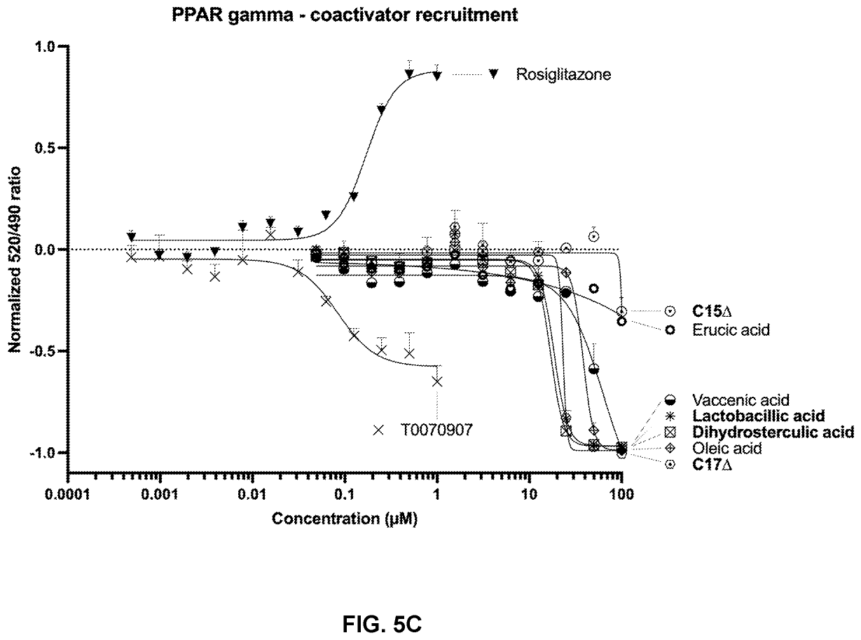

To identify agonist compounds, binding events of a number of CpFAs and fatty acids at the PPAR ligand binding domain were analyzed using Time-Resolved FRET (TR-FRET). The studies show the amount of ligand required to bind to receptor, effect a conformational change, and recruit coactivator peptide. These studies show several CpFAs bind the ligand binding domains of PPAR-α and PPAR-δ consistent with agonist activity (FIGS. 5A-B), whereas the results for PPAR-γ suggest a complex interaction of several CpFAs with PPAR-γ which alters the ligand binding domain of this receptor (FIG. 5C).

### Example 5

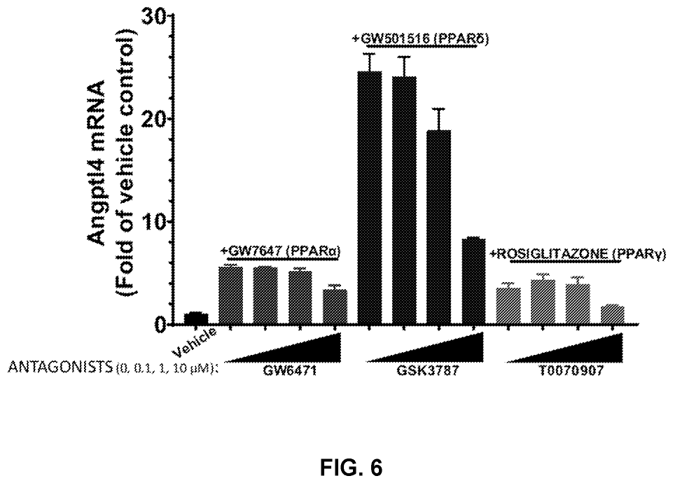

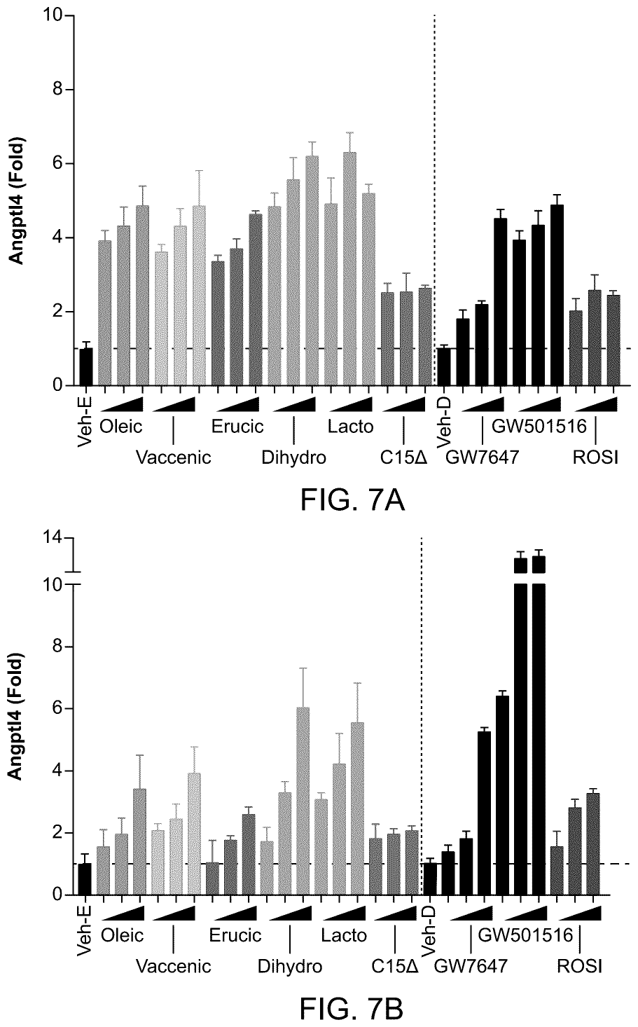

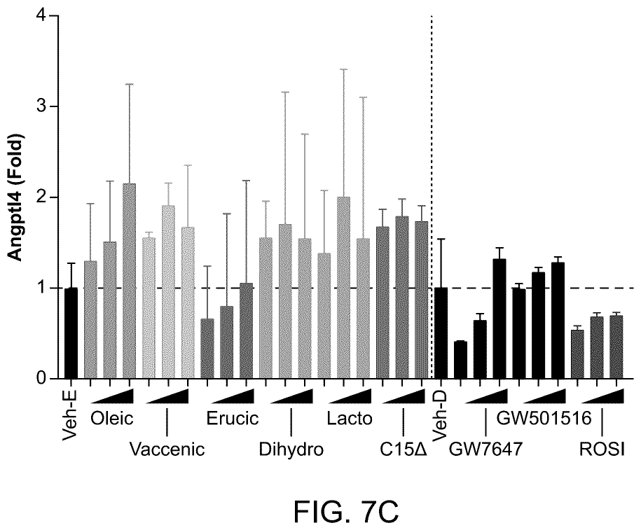

The angiopoietin-like 4 (Angptl4) gene can be upregulated via PPAR-induction. To study the effect of CpFAs on the regulation of Angptl4 expression, Angptl4 mRNA expression levels were quantified by real-time qPCR in cultured murine 3T3-L1 cells. FIG. 6 shows that the Angptl4 mRNA transcript provides a functional read-out of PPAR agonism and antagonism in living cells. FIG. 7A-C show a dose-dependent response to representative CpFAs in murine 3T3-L1 cells. These results indicate that Angptl4 expression can be induced by CpFAs in murine 3T3-LI cells.

### Methods

Docking was performed using Maestro from the Schrödinger suite (v12.8.117). Ligand structures were downloaded from PubChem, and human PPAR structures were downloaded from the Protein Data Bank and the AlphaFold database, when applicable. Glide-XP docking and Prime MM-GBSA postprocessing for every ligand-receptor pair were performed, and free binding energies (deltaG expressed in kcal/mol) were analyzed using R version 3.6.2.

The 2D chemical structures files were obtained from a public database (PubChem) for all ligands except cis-11,12-MPD and cis-13,14-MHD, which were translated from their SMILES structures using the NIH SMILES translator. For FIG. 4A, the following PDB crystallized structures were used for the docking: PPAR-α (1KKQ, 2P54), PPAR-δ (3TKM), PPAR-γ (3BC5, 5LSG, 5DWL, 3VSO). For FIG. 4B, the following PDB crystallized structures: PPAR-α (2ZNN, 3KDU, 3VI8, 4BCR, 6KAX, 6KB0, 6KB6, 6KB8, 6KBA), PPAR-δ (1Y0S, 2B50, 2Q5G, 3TKM, 5U3V, 5U3W, 5U46, 5ZXI, 6A6P), PPAR-γ (2Q5S, 2Q61, 3B0Q, 3B0R, 3BC5, 3WMH, 4A4V, 4EM9, 5TTO). For PPAR α, γ and δ, AI-generated AlphaFold structures (available via the world wide web at alphafold.ebi.ac.uk) were also used. For both FIGS. 4A and 4B, ligands and protein structures were prepared according to standard Schrödinger software protocols. Molecules sharing a letter are not significantly different from each other, at p<0.05 (ANOVA followed by post-hoc Tukey's Honest Significant Difference test).

Materials and Reagents: The TR-FRET binding studies were conducted utilizing the “LanthaScreen TR-FRET Coactivator Assay” suite of kits provided by ThermoFisher Scientific. Reference PV4684 was used for PPAR-α, reference PV4685 for PPARδ, and reference PV4548 for PPARγ. All non-CpFA high affinity PPAR agonists and antagonists were purchased from Cayman Chemicals. All lipids and CpFAs except for cis-11,12-MPD, cis-13,14-MHD and CPOA2H were also purchased from Cayman Chemicals. cis-11,12-MPD, cis-13,14-MHD and CPOA2H were custom-synthesized as a fee-for-service by Larodan AB.

Binding of PPAR sub-types with selected CpFAs: The procedures were consistent across all three TR-FRET kits. First, all ligands were prepared at a 100× concentration in 100% DMSO, and subsequently diluted in a 12-point dilution series in 100% DMSO. Each 100× agonist dilution was further diluted to 2× in the Complete Nuclear Receptor Buffer provided in the kits. The respective PPAR ligand binding domains (LBD) were prepared at a 4× concentration in cold Complete Nuclear Receptor Buffer. Additionally, solutions containing 500 nM of fluorescein bound to specific coactivator peptides for PPAR α, δ, and γ (PGC1a, C33, and TRAP220/DRIP-2, respectively), as well as 20 nM of Tb anti-GST antibody were prepared. All ligands and solutions were then added to separate 384-well assay plates in quadruplicate, incubated at room temperature, shielded from light, for 1 hour. Finally, the plates were read at wavelengths of 520 nm and 495 nm. Data were analyzed using GraphPad Prism 9.5.1. The 520/495 ratio was plotted against the ligands' concentrations in log scale. Non-linear curve fitting was done using the 4PL method in GraphPad Prism, as recommended by the manufacturer.

3T3-L1 cell culture and PPAR activation: 3T3-LI preadipocytes were cultured in growth medium (high glucose Dulbecco's Modified Eagle Medium (DMEM) containing 10% calf serum (CS), 1× Pencillin/Streptomycin, and 1× Glutamax) and seeded in multi-well (12-or 24-well) plates. PPAR specificity of gene target activation was determined in 3T3-L1 preadipocytes using well-described agonists and antagonists for PPAR-α, PPAR-δ, or PPAR-γ. On the day of treatment, preadipocytes were serum starved in 0.25% CS in growth medium for 2-4 hours. Cells were pretreated with increasing concentrations (0, 0.1, 1 and 10 μM) of PPAR antagonists (GW6471 for PPAR-α, GSK3787 for PPARδ, and T0070907 for PPARγ) in serum starve medium. After 1 hour, selective PPAR agonists were added (GW7647 (PPAR-α), GW501516 (PPAR-δ), and rosiglitazone (PPAR-γ)) and incubated for 24 hours. Lysates were collected for RNA extraction. To test if CpFAs demonstrate agonism of PPAR isoforms, 3T3-L1 preadipocytes were serum starved in 0.25% CS for 2-4 hours. CpFA treatments (final concentrations of 37.5, 75, and 150 μM) were prepared in 0.25% serum starve media plus 3% BSA, followed by heating (55° C.) and sonicating for 30 minutes. Treatments were allowed to cool to 37° C. and then added to 3T3-L1 preadipocytes and incubated for 24 hours. Lysates for RNA isolation were collected.

Angptl4 expression analyses: RNA was isolated from cell lysates using Qiagen 96 Total RNA kit. cDNA was synthesized and plated in triplicates in 384-well plates. RT-qPCR was performed using TaqMan Fast-Advanced master mix and TaqMan reagents and primers (18S, rplp0, angplt4) and analyzed using a Thermo Fisher Scientific QuantStudio 6 Flex PCR system. Relative gene expression was analyzed using the fold difference algorithm (2∧(−ΔΔCt)) using the geomean of 2 housekeeping genes (18S, RPLPO) compared to relevant vehicle control.

Other embodiments of the present disclosure are possible. Although the description above contains specific examples, these are presented for the purposes of illustration and description and should not be construed as limiting the scope of the disclosure. For example, combinations and sub-combinations of the specific features and aspects of the embodiments described above fall within the scope of this disclosure. Various features and aspects of the disclosed embodiments can be combined with or substituted for one another in order to form various embodiments. All structural, chemical, and functional equivalents to the elements of the above-described examples that are known to those of ordinary skill in the art are encompassed by the present claims. Thus, the scope the present disclosure should not be limited by the particular examples described above.

No element, component, or method step in the present disclosure is intended to be dedicated to the public regardless of whether the element, component, or method step is explicitly recited in the claims. Reference to an element in the singular is not intended to mean “one and only one” unless explicitly so stated, but rather “one or more.” As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising,” “consisting essentially of” and “consisting of” may be replaced with either of the other two terms. The examples illustratively described herein suitably may be practiced in the absence of any element or elements, limitation or limitations which is not specifically disclosed herein. The scope of this disclosure should be determined by the appended claims and their legal equivalents.

What is claimed is:

1. A compound of Formula I: 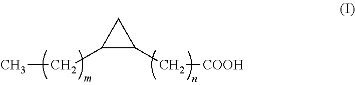
wherein m=2 and n is an odd-number between 4 and 20, or a derivative thereof, or a salt, or a prodrug thereof, for use as a medicament.

2. The compound of claim 1, for treatment or management of a PPAR-responsive condition.

3. The compound of claim 1 or 2, wherein the medicament is for treatment or management of weight disorders, insulin resistance, hyperinsulinemia, impaired glucose tolerance, metabolic disorders, diabetes in an obese individual, diabetic complication, a chronic inflammatory disorder, inflammatory bowel disease, ulcerative colitis, Crohn's disease, gastrointestinal disorders, gastroesophageal reflux, diverticulitis, gastrointestinal ulcers, arthritis, rheumatoid arthritis, polyarthritis, asthma, ocular inflammation, thy eye a skin disorder, psoriasis, cancer, liposarcoma, prostate cancer, cervical, breast, multiple myeloma, pancreatic cancer, neuroblastoma, bladder cancer, lipid disorders, hyperlipidemia, dyslipidemia diabetic dyslipidemia, mixed dyslipidemia, hypoalphalipoproteinemia, hypertriglyceridemia, hypercholesterolemia, low high density lipoprotein (HDL), fatty liver disease, non-alcoholic fatty liver disease, non-alcoholic steatohepatitis, cardiovascular disease, and atherosclerosis.

4. The compound of any one of claims 1 to 3, wherein n=9 or n=11.

5. The compound of any one of claims 1 to 4, wherein the compound of Formula I is cis-11,12-methylene-pentadecanoic acid (cis-11,12-MPD), cis-13,14-methylene-heptadecanoic acid (cis-13,14-MHD), or a combination thereof.

6. A method of treating or managing a Peroxisome Proliferator-Activated Receptor (PPAR)-responsive condition in a subject in need thereof, the method comprising administering to the subject a nutraceutical or pharmaceutical composition comprising:
a therapeutically effective amount of an isolated PPAR ligand selected from the group consisting of:
a compound of Formula I: 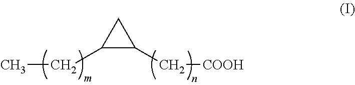
wherein m=2 and n is an odd-number between 4 and 20, or a derivative thereof, or a salt, or a prodrug thereof; and
a compound of Formula II: 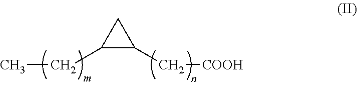
wherein m and n are independently an integer between 2 and 20, or a derivative thereof, or a salt, or a prodrug thereof, and combinations thereof; and
a nutraceutically or pharmaceutically acceptable carrier, excipient, adjuvant, or vehicle.

7. The method of claim 6, wherein the nutraceutical or pharmaceutical composition comprises the compound of Formula I, wherein n=9 or n=11.

8. The method of claim 7, wherein the compound of Formula I is cis-11,12-methylene-pentadecanoic acid (cis-11,12-MPD), cis-13, 14-methylene-heptadecanoic acid (cis-13,14-MHD), or a combination thereof.

9. The method of any one of claims 6 to 8, wherein the nutraceutical or pharmaceutical composition comprises the compound of Formula II, wherein m is 5 or 7 and n is an odd number between 7 and 17.

10. The method of claim 9, wherein the compound of Formula II is cis-9,10-methylene-hexadecanoic acid, lactobacillic acid, dihydrosterculic acid, or a combination thereof.

11. The method of any one of claims 6 to 10, wherein the nutraceutical or pharmaceutical composition is administered orally, topically, or parenterally.

12. The method of any one of claims 6 to 11, wherein the nutraceutical or pharmaceutical composition comprises about 1 mg to about 10 g of the isolated PPAR ligand.

13. The method of any one of claims 6 to 12, wherein the nutraceutical or pharmaceutical composition is administered once or four times per day.

14. The method of any one of claims 6 to 13, comprising administering the isolated PPAR ligand in a daily amount of from about 1 mg to about 10 mg, from about 1 mg to about 30 mg, from about 10 mg to about 100 mg, from about 100 mg to about 500 mg, from about 0.5 g to about 1 g, from about 1 g to about 2 g, from about 2 g to about 4 g, from about 4 g to about 6 g, or from about 6 g to about 10 g.

15. The method of any one of claims 6 to 14, wherein the PPAR-responsive condition is a PPARα-, PPARγ-or PPARδ-responsive condition.

16. The method of any one of claims 6 to 15, wherein the PPAR-responsive condition is selected from the group consisting of weight disorders, insulin resistance, hyperinsulinemia, impaired glucose tolerance, metabolic disorders, diabetes in an obese individual, diabetic complication, a chronic inflammatory disorder, inflammatory bowel disease, ulcerative colitis, Crohn's disease, gastrointestinal disorders, gastroesophageal reflux, diverticulitis, gastrointestinal ulcers, arthritis, rheumatoid arthritis, polyarthritis, asthma, ocular inflammation, dry eye a skin disorder, psoriasis, cancer, liposarcoma, prostate cancer, cervical, breast, multiple myeloma, pancreatic cancer, neuroblastoma, bladder cancer, lipid disorders, hyperlipidemia, dyslipidemia diabetic dyslipidemia, mixed dyslipidemia, hypoalphalipoproteinemia, hypertriglyceridemia, hypercholesterolemia, low blood high density lipoprotein (HDL), fatty liver disease, non-alcoholic fatty liver disease, non-alcoholic steatohepatitis, cardiovascular disease, and atherosclerosis.

17. The method of any one of claims 6 to 16, wherein the prodrug form of the PPAR ligand is administered.

18. A pharmaceutical composition for treating or managing a Peroxisome Proliferator-Activated Receptor (PPAR)-responsive condition in a subject in need thereof, the composition comprising: an isolated PPAR ligand of Formula I: 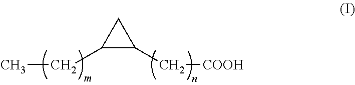
wherein m=2 and n is an odd-number between 4 and 20, or a derivative thereof, or a salt, or a prodrug thereof; and
a pharmaceutical carrier, excipient, adjuvant, or vehicle.

19. The composition of claim 18, wherein n=9 or n=11.

20. The composition of claim 19, wherein the isolated PPAR ligand of Formula I is cis-11,12-methylene-pentadecanoic acid (cis-11,12-MPD), cis-13,14-methylene-heptadecanoic acid (cis-13,14-MHD), or a combination thereof.

21. The composition of any one of claims 18 to 20, formulated for oral, topical, or parenteral administration.

22. The composition of any one of claims 18 to 21, formulated as a solid or liquid dosage form.

23. The composition of claim 22, wherein the dosage form is a tablet, coated tablet, capsule, powder, granulate, solution, dispersion, suspension, syrup, emulsion, or spray.

24. The composition of any one of claims 18 to 23, wherein the pharmaceutical carrier, excipient, adjuvant, or vehicle is selected from the group consisting of diluents, binders, fillers, extenders, humectants, tableting aids, disintegrating agents, absorption accelerators, absorbents, buffering agents, wetting agents, suspending agents, solvents, gel-forming agents, matrix-forming agents, solubilizing agents, emulsifiers, lubricants, encapsulating agents, delayed-release coatings, enteric coatings, sweetening agents, flavoring agents, fragrances, preservatives, and combinations thereof.

25. The composition of any one of claims 18 to 24, further comprising a compound of Formula II: 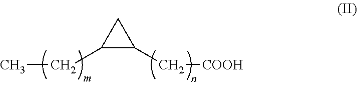
wherein m and n are independently an integer between 2 and 20, or a derivative thereof, or a salt, or a prodrug thereof.

26. The composition of any one of claims 18 to 25, further comprising a concomitant component as an additional active pharmaceutical ingredient.

27. The composition of claim 26, wherein the concomitant component is selected from the group consisting of therapeutic drugs for diabetes, therapeutic drugs for diabetic complications, therapeutic drugs for dyslipidemia, antihypertensive drugs, anti-obesity drugs, therapeutic drugs for fatty liver disease, and combinations thereof.

28. A method of treating or managing a Peroxisome Proliferator-Activated Receptor (PPAR)-responsive condition in a subject in need thereof, the method comprising administering to the subject a pharmaceutical composition according to any one of claims 18 to 27.

29. A nutraceutical supplement for promoting Peroxisome Proliferator-Activated Receptor (PPAR) modulation in a subject in need thereof, the supplement comprising:
an isolated PPAR ligand of Formula I: 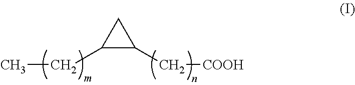
wherein m=2 and n is an odd-number between 4 and 20, or a derivative thereof, or a salt, or a prodrug thereof; and
a nutraceutically acceptable carrier, excipient, adjuvant, or vehicle.

30. The supplement of claim 29, wherein n=9 or n=11.

31. The supplement of claim 30, wherein the isolated PPAR ligand of Formula I is cis-11,12-methylene-pentadecanoic acid (cis-11,12-MPD), cis-13,14-methylene-heptadecanoic acid (cis-13,14-MHD), or a combination thereof.

32. The supplement of any one of claims 29 to 31, formulated for oral, topical, or parenteral administration.

33. The supplement of any one of claims 29 to 32, formulated as a tablet, coated tablet, capsule, aerosol, powder, granulate, suppository, solution, dispersion, suspension, syrup, emulsion, cream, ointment, gel, lotion, or spray.

34. The supplement of any one of claims 29 to 33, further comprising a concomitant component as an additional nutraceutical agent, wherein the concomitant component is selected from the group consisting of nutraceutical agents for antioxidant support of cells from damage caused by free radicals and oxidative stress; nutraceutical agents to support joint health; nutraceutical agents to improve joint mobility and reduce joint pain; nutraceutical agents to enhance cognitive function, memory, focus, or overall mental performance; nutraceutical agents to promote a healthy gut microbiome; nutraceutical agents to improve digestion and support overall gut health; nutraceutical agents to support healthy cholesterol levels; nutraceutical agents to promote heart health and reduce the risk of heart disease;
nutraceutical agents to boost energy levels, enhance vitality, or combat fatigue; nutraceutical agents to support immune system function or strengthen the body's natural defense mechanisms;
nutraceutical agents to support weight loss, boost metabolism, or reduce body fat; nutraceutical agents to promote healthy skin, reduce signs of aging, or improve skin elasticity; and combinations thereof.

35. The supplement of any one of claims 29 to 34, further comprising a compound of Formula II: 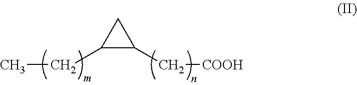
wherein m and n are independently an integer between 2 and 20, or a derivative thereof, or a salt, or a prodrug thereof.

36. A method of treating or managing an Angiopoetin-like 4 (Angptl4)-responsive condition in a subject in need thereof, the method comprising administering to the subject a nutraceutical or pharmaceutical composition comprising:
a therapeutically effective amount of an isolated cyclopropane fatty acid selected from the group consisting of:
a compound of Formula I: 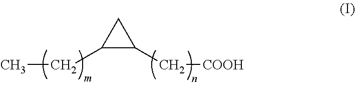
wherein m=2 and n is an odd-number between 4 and 20, or a derivative thereof, or a salt, or a prodrug thereof; and
a compound of Formula II: 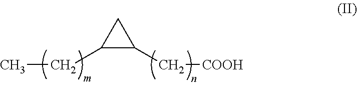
wherein m and n are independently an integer between 2 and 20, or a derivative thereof, or a salt, or a prodrug thereof, and combinations thereof; and
a nutraceutically or pharmaceutically acceptable carrier, excipient, adjuvant, or vehicle.

37. The method of claim 36, wherein the nutraceutical or pharmaceutical composition comprises a compound of Formula I, wherein n=9 or n=11.

38. The method of claim 37, wherein the compound of Formula I is cis-11,12-methylene-pentadecanoic acid (cis-11,12-MPD), cis-13,14-methylene-heptadecanoic acid (cis-13,14-MHD), or a combination thereof.

39. The method of claim of any one of claims 36 to 38, wherein the nutraceutical or pharmaceutical composition comprises the compound of Formula II, wherein m is 5 or 7 and n is an odd number between 6 and 18.

40. The method of claim 39, wherein the compound of Formula II is cis-9,10-methylene-hexadecanoic acid, lactobacillic acid, dihydrosterculic acid, or a combination thereof.

41. The method of any one of claims 36 to 40, wherein the nutraceutical or pharmaceutical composition is administered orally, topically, or parenterally.

42. The method of any one of claims 36 to 41, wherein the nutraceutical or pharmaceutical composition comprises about 1 mg to about 5 g of the isolated cyclopropane fatty acid.

43. The method of any one of claims 36 to 42, wherein the nutraceutical or pharmaceutical composition is administered once to four times per day.

44. The method of any one of claims 36 to 43, comprising administering the isolated cyclopropane fatty acid in a daily amount of from about 1 mg to about 10 mg, from about 1 mg to about 30 mg, from about 10 mg to about 100 mg, from about 100 mg to about 500 mg, from about 0.5 g to about 1 g, from about 1 g to about 2 g, from about 2 g to about 4 g, from about 4 g to about 6 g, or from about 6 g to about 10 g.

45. The method of any one of claims 36 to 44, wherein the Angpt14-responsive condition is selected from the group consisting of obesity, insulin resistance, diabetes, hypertriglyceridemia, and hepatic steatosis.

46. The method of any one of claims 36 to 45, wherein the prodrug form of the cyclopropane fatty acid is administered, or the nutraceutical or pharmaceutical composition further comprises the prodrug form of the compound of Formula I or Formula II.

47. A method of treating or managing an Angiopoetin-like 4 (Angpt14)-responsive condition in a subject in need thereof, the method comprising administering to the subject a pharmaceutical composition according to any one of claims 18 to 27.

48. A functional food or feed composition comprising:
a PPAR-modulating amount of a compound of Formula I: 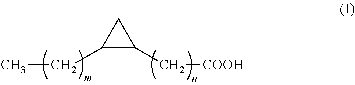
wherein m=2 and n is an odd-number between 4 and 20, or a derivative thereof, or a salt, or a prodrug thereof; and
an edible carrier.

49. The composition of claim 48, wherein n=9 or n=11.

50. The composition of claim 49, wherein the compound of Formula I is cis-11,12-methylene-pentadecanoic acid (cis-11,12-MPD), cis-13,14-methylene-heptadecanoic acid (cis-13,14-MHD), or a combination thereof.

51. The composition of any one of claims 48 to 50, comprising the prodrug form of the compound of Formula I.

52. The composition of any one of claims 48 to 51, wherein the composition includes about 50 mg to about 2 g of the compound of Formula I per serving.

53. The composition of any one of claims 48 to 52, further comprising a compound of Formula II: 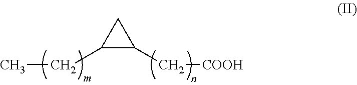
wherein m and n are independently an integer between 2 and 20, or a derivative thereof, or a salt, or a prodrug thereof.

54. The composition of any one of claims 48 to 53, wherein the composition is a food suitable for human consumption.

55. The composition of claim 54, wherein the food is a solid, semi-solid or liquid food stuff.

56. The composition of any one of claims 48 to 55, wherein the composition is a feed suitable for animal consumption.

57. The composition of any one of claims 48 to 56, further comprising an additional nutraceutical agent selected from the group consisting of caffeine, amino acids, creatine, glucosamine, chondroitin, collagen, essential fatty acids, antioxidants, vitamins, minerals, essential oils, extracts, prebiotics, and probiotics, and combinations thereof.

58. The composition of any one of claims 48 to 57, further comprising a concomitant component as an active pharmaceutical ingredient.

59. The composition of claim 58, wherein the concomitant component is selected from the group consisting of therapeutic drugs for diabetes, therapeutic drugs for diabetic complications, therapeutic drugs for dyslipidemia, antihypertensive drugs, anti-obesity drugs, therapeutic drugs for fatty liver disease, and combinations thereof.

60. A method of treating or managing an Angiopoetin-like 4 (Angptl4)-responsive or a Peroxisome Proliferator-Activated Receptor (PPAR)-responsive condition in a subject in need thereof, the method comprising administering to the subject a nutraceutical supplement according to any one of claims 29 to 35 or a functional food or feed composition according to any one of claims 48 to 59.
